(19) United States
(12) Patent Application Publication
(10) Pub. No.: US 2023/0065420 A1
(43) Pub. Date: Mar. 2, 2023

(54) COMPOUNDS AND METHODS FOR THE DIAGNOSIS, IMAGING AND TREATMENT OF NEURODEGENERATIVE DISEASES AND DISORDERS

(71) Applicant: The University of Melbourne, Melbourne (AU)

(72) Inventors: Paul Stephen Donnelly, Melbourne (AU); Katherine Anne Morgan, Melbourne (AU)

(73) Assignee: The University of Melbourne, Melbourne (AU)

(21) Appl. No.: 17/784,588

(22) PCT Filed: Dec. 11, 2020
(86) PCT No.: PCT/AU2020/051358

§ 371 Date: Jun. 10, 2022

(30) Foreign Application Priority Data
Dec. 12, 2019 (AU) 2019904711

(51) Int. Cl. A61K 51/10; C07D 257/08; C07F 1/08; A61P 25/00; G01N 33/68

(52) U.S. Cl. CPC A61K 51/1018; C07D 257/08; C07F 1/08; A61P 25/00; G01N 33/6896; C07B 2200/13

(57) ABSTRACT

The present invention relates to methods of diagnosis and therapy for neurodegenerative diseases and disorders.

### FIELD

The present invention relates to methods of diagnosis and therapy for neurodegenerative diseases and disorders.

### BACKGROUND

Neurodegenerative diseases and disorders encompass a range of conditions where the cells of the brains, for example, neurons, are affected. In some cases, cell death occurs and this results in neurodegeneration and the subsequent reduction or loss of communication with other cells and parts of the body. In other cases, neurodegeneration itself results in cell death and leads to the loss of cognitive function, memory and movement.

An example of a neurodegenerative disorder is Alzheimer's disease, which is typically characterised by the presence of the peptide amyloid beta (Aβ) in plaques or aggregations. Diagnosis of Alzheimer's disease is by confirmation of Aβ plaque formation post mortem. Presently, there is an inability to establish a definitive correlation between the plaque burden and any observed cognitive decline of the patient. Other neurodegenerative diseases and disorders are also typically characterised by the presence of a characteristic protein or peptide.

There are often difficulties associated with the imaging and diagnosis of neurodegenerative diseases and disorders. Since these diseases and disorders affect the brain and characteristic pathologies are localised therein, compounds and markers that may signify the disease must first be capable of entering the brain. While positron emission tomography (PET), single photon emission computed tomography (SPECT) and other imaging techniques have been used in an attempt to image, diagnose and potentially treat neurodegenerative diseases and disorders, these nuclear medicine techniques rely on a complex bearing the requisite radioisotope having favourable stability, decay characteristics, half-life and accumulation at the site of interest.

There remains a need for new compounds and methods for the diagnosis, imaging and treatment of neurodegenerative diseases and disorders. The compounds must be sufficiently stable, capable of coordinating a radioisotope and have the correct physical characteristics for localisation and accumulation in the brain.

### SUMMARY OF THE INVENTION

The present invention relates to methods for the diagnosis and/or therapy of neurodegenerative diseases and disorders. The present inventors have found that the administration of a modified antibody that is able to localise at the site of the disease or disorder, followed by a compound that contains a radioisotope and is specifically functionalised to react to the modified portion of the modified antibody, allows for improved visualisation and treatment of neurodegenerative diseases and disorders.

Accordingly, the present invention provides a method for the in vivo diagnostic imaging of a neurodegenerative disease or disorder, the method comprising sequentially administering to a patient:
i) an antibody associated with the neurodegenerative disease or disorder, which has been modified to comprise a click-receptive dienophile; and
ii) a compound of Formula (I) or a pharmaceutically acceptable salt thereof;

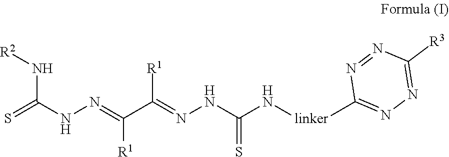

wherein:
each R¹ is independently selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R² is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R³ is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl; and
the linker is selected from the group consisting of optionally substituted alkylene, optionally substituted alkenylene, optionally substituted alkynylene, optionally substituted arylene, optionally substituted benzylene and heteroarylene; and

wherein the compound of Formula (I) is further complexed with a radioisotope and wherein the modified antibody covalently reacts in a click type reaction with the tetrazine of the compound of Formula (I) in situ.

In an embodiment, and with reference to this above aspect, the neurodegenerative disease or disorder is characterised by the presence of a protein aggregate associated with the neurodegenerative disease or disorder.

Given the nature of neurodegenerative diseases and disorders, the physiological sites implicated in these diseases and disorders are often in the brain. This means that any compounds used in the diagnosis or treatment of such diseases and disorders must first cross the blood brain barrier in order to reach the target site. Subsequently, these compounds must have the correct physical characteristics such that they are sufficiently lipophilic for transport across the blood brain barrier.

Antibodies that are associated with a given neurodegenerative disease or disorder will inherently have the physical features that allow for movement across the blood brain barrier. The present inventors have found that these antibodies may be modified in such a way so as to allow installation of a dienophile on the surface of the antibody while also maintaining the physical characteristics of the antibody overall for passage across the blood brain barrier.

Without wishing to be bound by theory, the inventors presently believe that selecting an antibody or an antibody fragment that is associated with a neurodegenerative disease or disorder allows for binding and localisation of the antibody at a site that is implicated in the disease or disorder. The modified antibody containing a dienophile that is the subject of the present invention is capable of undergoing a click-type reaction with a suitable reaction partner. An example of a suitable reaction partner is a diene or similar. Where a dienophile is in the presence of a diene, a cycloaddition reaction, for example, results in the formation of new covalent bonds. The compound of Formula (I) that is subsequently administered contains a tetrazine moiety that can undergo a cycloaddition reaction with the dienophile that is installed on the modified antibody. Once administered, the compound of Formula (I) moves through the circulation until it reaches the site at which the antibody is located. Since the dienophile of the antibody and the tetrazine of Formula (I) are in close proximity and the cycloaddition reaction (i.e. the click-type reaction) between these groups is kinetically favoured, the cycloaddition reaction proceeds with the formation of new covalent bonds. This results in the attachment of the compound of Formula (I) to the site associated with the neurodegenerative disease or disorder, via the antibody.

The methods disclosed herein may be considered to constitute a “pre-targeting approach” to diagnostic imaging and treatment. The present inventors have found that the initial administration of a modified antibody containing, for instance, a trans-cyclooctene functional group allows the modified antibody to be concentrated at its target site (i.e. the site at which the protein aggregate associated with the neurodegenerative disease or disorder is present). Where administration of the antibody is chronic, i.e. over a period of a month or so, a steady state of the antibody in vivo is obtained and the target site is “pre-targeted”. Once the requisite concentration of the modified antibody is obtained, the compound of Formula (I) containing the tetrazine functional group is administered. Given the bioorthogonal nature of the reaction between the tetrazine and the TCO group, the click reaction occurs quickly and the compound of Formula (I) containing the radioisotope is readily localised. Since the concentration of the modified antibody is sufficiently high, the radioisotope (as coordinated to the compound of Formula (I)) is also increased, when compared to a standard radioimaging approach (i.e. where a complexed radioisotope is simply administered). This allows for images of greater contrast and better quality to be obtained, which in turn provides for greater confidence in the diagnosis of the neurodegenerative disease.

In an embodiment, and with reference to this above aspect, the protein aggregate consists of an amyloid-beta plaque. In a further embodiment, the protein aggregate consists of a soluble amyloid-beta oligomer.

In another embodiment, and with reference to this above aspect, the protein aggregate consists of a neurofibrillary tangle.

In another embodiment, and with reference to this above aspect, the protein aggregate consists of a hyperphosphorylated tau protein.

In another embodiment, and with reference to this above aspect, the protein aggregate consists of a Lewy body.

In another embodiment, and with reference to this above aspect, the protein aggregate consists of aggregated alpha-synuclein.

In another embodiment, and with reference to this above aspect, the protein aggregate consists of aggregated TDP-43 protein.

In an embodiment, and with reference to this above aspect, the neurodegenerative disease or disorder is Alzheimer's disease.

In another embodiment, and with reference to this above aspect, the neurodegenerative disease or disorder is a brain cancer. In an embodiment, the brain cancer is a brain metastasis. In an embodiment, the brain metastasis is related to a breast cancer. In an embodiment, the brain cancer is glioblastoma multiforme or meningioma.

The antibodies discussed in the present invention show an affinity for the protein aggregate that is characteristic of a neurodegenerative disease or disorder.

In an embodiment, and with reference to this above aspect, the click-receptive dienophile on the modified antibody is a trans-cyclooctene (TCO) residue.

In an embodiment, and with reference to this above aspect, the modified antibody is an antibody selective for an amyloid-beta plaque. Preferably, the antibody is selected from 6E10 (eg, IgG, purified monoclonal anti-beta-amyloid, 1-16 antibody) which is reactive to aa1-16 Abeta and to APP, and 1E8 (IgG1, kappa) (see Nagasaki, et al. Pathology International Vol 45., Issue 4, April 1995, pp 266-274), and are modified according to the invention described herein.

In an embodiment, and with reference to this above aspect, the modified antibody is an antibody selective for a neurofibrillary tangle.

In an embodiment, and with reference to this above aspect, the modified antibody is an antibody selective for a hyperphosphorylated tau protein.

In another embodiment, and with reference to this above aspect, the modified antibody is an antibody specific for a Lewy body.

In another embodiment, and with reference to this above aspect, the modified antibody is an antibody specific for a TDP-43 protein or a TDP-43 aggregate.

In another embodiment, and with reference to this above aspect, the neurodegenerative disease or disorder is Alzheimer's disease.

In another embodiment, and with reference to this above aspect, the modified antibody is an antibody specific for a brain cancer.

In an embodiment, and with reference to this above aspect, the radioisotope is ⁶⁴Cu or ⁶⁷Cu.

In an embodiment, and with reference to this above aspect, the method involves the ex vivo complexation of the radioisotope with the compound of Formula (I) prior to in vivo administration.

In a further aspect the invention also provides a method for the ex vivo diagnostic imaging of a neurodegenerative disease or disorder, the method comprising sequentially administering to a cell sample:
i) an antibody associated with the neurodegenerative disease or disorder, which has been modified to comprise a click-receptive dienophile; and
ii) a compound of Formula (I) or a pharmaceutically acceptable salt thereof;

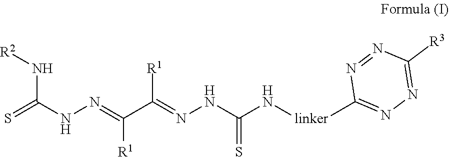

wherein:
each R¹ is independently selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R² is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R³ is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl; and
the linker is selected from the group consisting of optionally substituted alkylene, optionally substituted alkenylene, optionally substituted alkynylene, optionally substituted arylene, optionally substituted benzylene and heteroarylene; and

wherein the compound of Formula (I) is further complexed with a radioisotope and wherein the antibody covalently reacts with the tetrazine of the compound of Formula (I) in a click-type reaction.

In an embodiment, the neurodegenerative disease or disorder is characterised by the presence of a protein aggregate associated with the neurodegenerative disease or disorder.

In an embodiment, and with reference to this above aspect, the neurodegenerative disease or disorder is Alzheimer's disease (AD).

In another embodiment, and with reference to this above aspect, the neurodegenerative disease or disorder is a brain cancer. In an embodiment, the brain cancer is a brain metastasis. In an embodiment, the brain metastasis is related to a breast cancer. In an embodiment, the brain cancer is glioblastoma multiforme or meningioma.

In an embodiment, and with reference to this above aspect, the click-receptive dienophile is a trans-cyclooctene (TCO) residue.

In an embodiment, and with reference to this above aspect, the modified antibody is an antibody selective for an amyloid-beta plaque. Preferably, the antibody is selected from 6E10 and 1E8 and is modified accordingly.

In an embodiment, and with reference to this above aspect, the modified antibody is an antibody selective for a neurofibrillary tangle.

In an embodiment, and with reference to this above aspect, the modified antibody is an antibody selective for a hyperphosphorylated tau protein.

In another embodiment, and with reference to this above aspect, the modified antibody is an antibody selective for a Lewy body or alpha-synuclein.

In another embodiment, and with reference to this above aspect, the modified antibody is an antibody selective for a TDP-43 protein.

In another embodiment, and with reference to this above aspect, the modified antibody is an antibody selective for a brain cancer.

In an embodiment, and with reference to this above aspect, the radioisotope is a radioisotope of copper (Cu), technetium (Tc), gallium (Ga) or cobalt (Co). In an embodiment, the radioisotope is an isotope of copper. In another embodiment, the radioisotope is ⁶⁰Cu, ⁶¹Cu, ⁶²Cu, ⁶⁴Cu, ⁶⁷Cu, ^{99m}Tc, ⁶⁸Ga, ⁵⁵Co or ⁴⁴Sc.

In another aspect, the present provides a method for the treatment of a neurodegenerative disease or disorder, the method comprising sequentially administering to a patient an effective amount of:
i) an antibody associated with the neurodegenerative disease or disorder which has been modified to comprise a click-receptive dienophile; and
ii) a compound of Formula (I) or a pharmaceutically acceptable salt thereof;

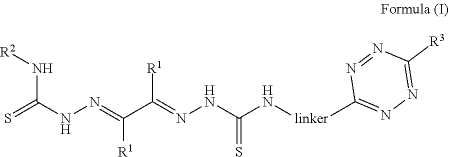

wherein:
each R¹ is independently selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R² is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R³ is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl; and
the linker is selected from the group consisting of optionally substituted alkylene, optionally substituted alkenylene, optionally substituted alkynylene, optionally substituted arylene, optionally substituted benzylene and heteroarylene,

wherein the compound of Formula (I) is further complexed with a radioisotope and wherein the modified antibody covalently reacts in a click-type reaction with the tetrazine of the compound of Formula (I) in situ.

In another embodiment, and with reference to this above aspect, the neurodegenerative disease or disorder is a brain cancer. In an embodiment, the brain cancer is a brain metastasis. In an embodiment, the brain metastasis is related to a breast cancer. In an embodiment, the brain cancer is glioblastoma multiforme or meningioma.

In an embodiment, the neurodegenerative disease or disorder is characterised by the presence of a protein aggregate associated with the neurodegenerative disease or disorder.

In an embodiment, and with reference to this above aspect, the protein aggregate consists of a amyloid-beta plaque. In another embodiment, the protein aggregate consists of a soluble amyloid-beta oligomer.

In another embodiment, and with reference to this above aspect, the protein aggregate consists of a neurofibrillary tangle.

In another embodiment, and with reference to this above aspect, the protein aggregate consists of a hyperphosphorylated tau protein.

In another embodiment, and with reference to this above aspect, the protein aggregate consists of a Lewy body.

In another embodiment, and with reference to this above aspect, the protein aggregate consists of a TDP-43 protein.

In an embodiment, and with reference to this above aspect, the neurodegenerative disease or disorder is Alzheimer's disease.

In another embodiment, and with reference to this above aspect, the neurodegenerative disease or disorder is a brain cancer. In an embodiment, the brain cancer is glioblastoma multiforme or meningioma.

In still a further aspect, the present invention provides a compound of Formula (I), or a pharmaceutically acceptable salt thereof:

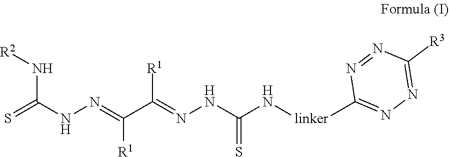

wherein:
each R¹ is independently selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R² is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R³ is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl; and
the linker is selected from the group consisting of optionally substituted alkylene, optionally substituted alkenylene, optionally substituted alkynylene, optionally substituted arylene, optionally substituted benzylene and optionally substituted heteroarylene.

The compounds of Formula (I) contain a bis(thiosemitcarbazone) functionality that is capable of coordinating a metal ion, for example, a copper (Cu^{II/I}) ion. The complex of Formula (I) with a copper ion is tetradentate in nature with the sulfur atoms and two of the nitrogen atoms coordinated to the copper ion. The present inventors have found that compounds of Formula (I) are ligands capable of forming strong complexes with copper ions. Where the copper ion is a radionuclide and therefore capable of undergoing radioactive decay, minimising the loss of the copper ion from the complex to other areas is favourable. This then minimises unwanted damage from exposure to radioactivity at other sites.

As the compounds of Formula (I) are intended to react with the modified antibody administered prior, the compounds of Formula (I) must also be able to cross the blood brain barrier. This means that compounds of Formula (I) must be sufficiently lipophilic and are limited in relation to the functional groups that may be present as part of the compound. The nature of the variable groups R¹, R² and R³ and the linker joining the tetrazine to the bis(thiosemicarbazone) functional group in the compounds of Formula (I) influence the overall lipophilicity of the compound. The present inventors have found that small non-polar groups, such as short-chain alkyl groups at variables R¹, R² and R³ provide compounds of Formula (I) with the requisite lipophilicity and subsequently, physical characteristics, to allow crossing of the blood brain barrier. Additionally, linkers such as small aromatic groups between the tetrazine and bis(thiosemicarbazone) also contribute to the required physical characteristics. Since the compounds of Formula (I) are intended to react with a modified antibody comprising a click-reactive dienophile, the steric and electronic features of the tetrazine should be optimised in order to facilitate a click-type reaction that is sufficiently fast. The kinetics of the click-type reaction will be influenced by the substituent for the R³ variable and also to some extent, the nature of the linker group. In addition, the compounds of Formula (I) must display the requisite metabolic stability and ability to coordinate and transport the coordinated metal ion, as the compounds are administered to patients and are subjected to physiological conditions. Again, the nature of the groups at variables R¹, R² and R³ and the linker group influence the metabolic stability of the compound (and eventual complex) and ability of the compound to coordinate and retain a metal ion.

In an embodiment, the present invention also provides a metal complex of compounds of Formula (I) having the structure of Formula (Ia):

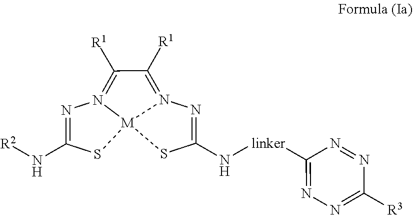

wherein M is a metal ion and R¹, R², R³ and the linker group are as defined above.

In an embodiment, and with reference to this above aspect, the compound of Formula (I) or salt thereof is presented as a radiolabelled complex, preferably complexed with a radioisotope such as ⁶⁴Cu or ⁶⁷Cu.

In an embodiment, and with reference to this above aspect, each occurrence of R¹ is methyl and R² is methyl.

In an embodiment, and with reference to this above aspect, each occurrence of R¹ is ethyl and R² is ethyl.

In an embodiment, and with reference to this above aspect, R³ is H.

In an embodiment, and with reference to this above aspect, R³ is methyl.

In an embodiment, and with reference to this above aspect, the linker is an arylene group, preferably a phenylene or a benzylene group.

In an additional aspect, the present invention provides a composition comprising a compound of Formula (I), or a pharmaceutically acceptable salt thereof, together with one or more pharmaceutically acceptable excipients.

In a further aspect, the present invention also provides a modified antibody having the structure of Formula (II):

Ab-linker-click-reactive dienophile Formula (II)

wherein:
Ab represents an antibody; and
the linker comprises one or more ethylene glycol units.

In an embodiment, the linker comprises more than one ethylene glycol units and represents a polyethylene glycol group.

In a further embodiment, the present invention provides a modified antibody having the structure of Formula (II), wherein the antibody is associated with a neurodegenerative disease or disorder or a brain cancer. In an embodiment, the click-reactive dienophile in the modified antibody of Formula (II) is a trans-cyclooctene (TCO) group.

The present inventors have found that the surface of an antibody may be modified to incorporate a TCO functionality by linking the functional group using to the antibody via one or more ethylene glycol units. This provides an antibody that retains its inherent function as an antibody, yet comprises a functional group that can react under click-type reactions. The linking group between the antibody and the TCO group may comprise one or more ethylene glycol units. Where the linking group comprises more than one ethylene glycol unit, the linking group may be considered as a polyethylene glycol group. The present inventors have found that the distance between the TCO functional group and the antibody itself is affected by the length of the ethylene glycol linking group, where the longer the linking group, the greater the distance is between the antibody and the TCO functional group. It is also important that the length of the linking group is such that the activity of the antibody when modified as described herein is maintained and also that the attached TCO functional group is sufficiently unhindered for participation in a click-type reaction.

In an embodiment, and with reference to this above aspect, the antibody is selective for a protein aggregate that is associated with a neurodegenerative disease or disorder or a brain cancer. In an embodiment, the brain cancer is a brain metastasis. In an embodiment, the brain metastasis is related to a breast cancer.

In an embodiment, and with reference to this above aspect, the click-receptive dienophile is a trans-cyclooctene (TCO) group.

In an embodiment, and with reference to this above aspect, the antibody is an antibody selective for an amyloid-beta plaque. Preferably, the antibody is selected from 6E10 or 1E8 and is modified accordingly.

In an embodiment, and with reference to this above aspect, the modified antibody is an antibody selective for a neurofibrillary tangle.

In an embodiment, and with reference to this above aspect, the modified antibody is an antibody selective for a hyperphosphorylated tau protein.

In another embodiment, and with reference to this above aspect, the modified antibody is an antibody selective for a Lewy body.

In another embodiment, and with reference to this above aspect, the modified antibody is an antibody selective for a TDP-43 protein.

In another embodiment, the present invention provides a modified antibody having the structure of Formula (II), wherein the antibody is associated with a brain cancer.

In another embodiment, the modified antibody is an antibody selective for a brain cancer. Preferably, the brain cancer is glioblastoma multiforme or meningioma.

### BRIEF DESCRIPTION OF THE FIGURES

Aspects and embodiments of the present disclosure are described herein, by way of non-limiting example only, with reference to the following drawings.

FIG. 1: The ORTEP representation of [CuL³]. Thermal ellipsoids of non-hydrogen atoms are represented at 30% probability. Hydrogen atoms and DMSO molecule are omitted for clarity. Selected bond lengths and crystallographic information for [CuL³] are also provided.

FIG. 2: Cyclic voltammograms of [CuL³] (1 mM) in DMF (versus. Ag/Ag+, referenced to Fc/Fc+ couple E^{o′}=0 V) (Bu)₄NPF₆ supporting electrolyte (0.1M).

FIG. 3: Cyclic voltammograms of [CuL⁴] (1 mM) in DMF (versus Ag/Ag+, referenced to Fc/Fc+ couple E^{o′}=0 V) (Bu)₄NPF₆ supporting electrolyte (0.1M).

FIG. 4: Cyclic voltammograms of (a) H₂L³ and (b) H₂L³ (1 mM) in DMF (versus Ag/Ag+, referenced to Fc/Fc+ couple E^{o′}=0 V) (Bu)₄NPF₆ supporting electrolyte (0.1M).

FIG. 5: Radio-HPLC chromatogram of [⁶⁴Cu[CuL³] (red, scintillation detector) and [CuL³] (blue, λ=350 nm). Signals were normalised and [CuL³] offset by 2 minutes for clarity.

FIG. 6: SEC-ICP-MS of click reaction with 1E8 antibody and [CuL³]. Unmodified 1E8 antibody incubated with an excess of [CuL³] (blue), 1E8-PEG4-TCO incubated with an excess of [CuL³] (red).

FIG. 7: ESI-TOF deconvoluted mass spectra of (a) unmodified Herceptin (HER); (b) HER-PEG₄-TCO showing between 1-4 TCO attachments; (c) HER-PEG₄-TCO reacted with [CuL³] and (d) HER-PEG4-TCO reacted with [CuL²]. Both (c) and (d) show complete reaction between the TCO functional groups and tetrazine containing copper complexes.

FIG. 8: IHC staining of serial AD brain sections (7 μm). (a) 1E8 IHC stained Aβ+ plaques, (b) and (c) 6E10-PEG₄-TCO, shows addition of TCO functional groups do not interfere with antibody binding to both diffuse and dense plaques. Scale bar=100 μm.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

The articles “a” and “an” are used herein to refer to one or to more than one (i.e. to at least one) of the grammatical object of the article. By way of example, “an element” means one element or more than one element.

In the context of this specification, the term “about” is understood to refer to a range of numbers that a person skilled in the art would consider equivalent to the recited value in the context of achieving the same function or result.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

Use of the term “associated with” herein describes a temporal, physical or spatial relationship between events, symptoms or pathologies. Thus for example, in the context of the present disclosure, an antibody associated with a neurodegenerative disease or disorder means that the neurodegenerative disease or disorder at least partially characterized by, either directly or indirectly, the accumulation, typically extracellular accumulation of such an antibody.

As used herein the terms “treating”, “treatment”, and grammatical equivalents refer to any and all uses which remedy the stated neurodegenerative disease or disorder, prevent, retard or delay the establishment of the disease or disorder, or otherwise prevent, hinder, retard, or reverse the progression of the disease or disorder. Thus the terms “treating” and the like are to be considered in their broadest context. For example, treatment does not necessarily imply that a patient is treated until total recovery. Where the disease displays or is characterized by multiple symptoms, the treatment or prevention need not necessarily remedy, prevent, hinder, retard, or reverse all of said symptoms, but may prevent, hinder, retard, or reverse one or more of said symptoms.

As used herein the term “effective amount” includes within its meaning a non-toxic but sufficient amount or dose of an agent or compound to provide the desired effect. The exact amount or dose required will vary from subject to subject depending on factors such as the species being treated, the age, size, weight and general condition of the subject, the severity of the disease or condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

As used herein, the term “pharmaceutically acceptable salts” refers to salts that retain the desired biological activity of the parent compound, and include pharmaceutically acceptable acid addition salts and base addition salts. Suitable pharmaceutically acceptable acid addition salts of compounds of Formula (I) may be prepared from an inorganic acid or an organic acid. Examples of an inorganic acid include hydrochloric acid, sulphuric acid and phosphoric acid. Examples of organic acids include aliphatic, cycloaliphatic, aromatic, heterocyclic carboxylic and sulfonic organic acids, such as, formic, acetic, proprionic, succinic, glycolic, gluronic, lactic, malic, tartaric, citric, fumaric, maleic, alkylsulfonic and arylsulfonic acids. Where the compound of Formula (I) is a solid, the compounds and salts thereof may exist in one or more different crystalline or polymorphic forms, all of which are intended to be within the scope of Formula (I).

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a human.

As used herein, the term “antibody” is used in the broadest sense and specifically covers intact antibodies, monoclonal antibodies, polyclonal antibodies, multispecific antibodies (e.g. bispecific antibodies) formed from at least two intact antibodies, and antibody fragments, so long as they exhibit the desired biological activity. The term refers to a molecule that has binding affinity for a target. It will be understood that this term extends to immunoglobulins, immunoglobulin fragments and non-immunoglobulin derived protein frameworks that exhibit antigen-binding activity. Representative antigen-binding molecules that are useful in the practice of the present invention include polyclonal and monoclonal antibodies as well as their fragments (such as Fab, Fab′, F(ab′)2, Fv), single chain (scFv) and domain antibodies (including, for example, shark and camelid antibodies), and fusion proteins comprising an antibody, and any other modified configuration of the immunoglobulin molecule that comprises an antigen binding/recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known. Antigen-binding molecules also encompass dimeric antibodies, as well as multivalent forms of antibodies. In some embodiments, the antibodies are chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Also contemplated, are humanized antibodies, which are generally produced by transferring complementarity determining regions (CDRs) from heavy and light variable chains of a non-human (e.g. rodent, preferably mouse) immunoglobulin into a human variable domain. Typical residues of human antibodies are then substituted in the framework regions of the non-human counterparts. The use of antibody components derived from humanized antibodies obviates potential problems associated with the immunogenicity of non-human constant regions.

A neurodegenerative disease or disorder manifests as the progressive loss of neurological structure and function. Examples of such diseases and disorders include Alzheimer's disease, Parkinson's disease, Huntington's disease and amyotrophic lateral sclerosis (i.e. motor neurone disease). These diseases are typically progressive, incurable and result in the loss of memory, cognition and movement in the patient. The hallmarks of many neurodegenerative diseases and disorders include abnormal protein aggregation.

As used herein, the term “Alzheimer's disease” is used to refer to the disease that is thought to be characterised by the presence of extracellular amyloid-beta (Aβ) plaques. It is thought that formation of Aβ plaques in the brain tissue is a primary determinant of Alzheimer's disease. Aβ is a 39 to 43 amino acid peptide fragment derived from the larger amyloid precursor protein (APP). The presence of Aβ plaques leads to the aggregation of soluble protofibrils, oligomers and senile Aβ plaques. This results in inflammation, oxidative stress, synaptic dysfunction and neuronal loss in the brain.

Other proteins implicated in neurodegenerative diseases and disorders include a hyperphosphorylated tau protein, a Lewy body, alpha-synuclein, huntingtin and TDP-43.

As used herein, the term “alkyl” refers to monovalent alkyl groups that may be straight chained or branched, and preferably have from 1 to 10 carbon atoms, or more preferably 1 to 6 carbon atoms. Examples of such groups include methyl, ethyl, n-isopropyl, iso-propyl, n-butyl, iso-butyl, n-hexyl, and the like. The alkyl group may be further optionally substituted.

As used herein, the term “alkenyl” refers to a monovalent aliphatic group having at least one carbon-carbon double bond and which may be straight chained or branched, preferably having from 2 to 10 carbon atoms. Examples of such groups include a vinyl or ethenyl group (—CH═CH₂), n-propenyl (—CH₂CH═CH₂), iso-propenyl (—C(CH₃)═CH₂), but-2-enyl (—CH₂CH═CHCH₃), and the like. The alkyl group may be further optionally substituted.

As used herein, the term “alkynyl” refers to a monovalent aliphatic group having at least one carbon-carbon triple bond and which may be straight chained or branched, preferably having from 2 to 10 carbon atoms. Examples of such groups include an acetylene or ethynyl group (—C≡CH), propargyl (—CH₂C≡CH), and the like.

As used herein, the term “heteroalkyl” refers to a monovalent straight- or branched chain alkyl group, preferably having from 2 to 12 carbon atoms, in which one or more of the carbon atoms (and any associated hydrogen atoms) are each independently replaced by a heteroatomic group selected from S, O, P and N.

As used herein, the term “cycloalkyl” refers to a saturated monocyclic or fused spirocyclic spiro polycyclic, carbocycle preferably containing from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. A cycloalkyl group typically is a C₃-C₉ cycloalkyl group.

As used herein, the term “aryl” refers to “a group or part of a group denoting (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. Typically an aryl group is a C₆-C₁₈ aryl group.

As used herein, the term “heteroaryl” refers to a group containing an aromatic ring (preferably a 5 or 6 membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. A heteroaryl group is typically a C₃-C₁₈ heteroaryl group.

As used herein, the term “alkylene” refers to divalent alkyl groups preferably having from 1 to 12 carbon atoms and more preferably 1 to 6 carbon atoms, and even more preferably 1 to 3 carbon atoms. Examples of such alkylene groups include methylene (—CH₂—), ethylene (—CH 2CH₂—), and the propylene isomers (e.g., —CH₂CH₂CH₂— and —CH(CH₃)CH₂—), and the like. In a similar manner, the terms “phenylene” and “benzylene” refer to divalent phenyl and benzyl groups respectively.

As used herein, the term “optionally substituted” in relation to a particular group is taken to mean that the group may or may not be further substituted with one or more groups selected from hydroxyl, acyl, alkyl, alkoxy, alkenyl, alkenyloxy, alkynyl, alkynyloxy, amino, aminoacyl, alkylaryl, aryl, aryloxy, carboxyl, acylamino, cyano, halogen, nitro, sulphate, phosphate, phosphine, heteroaryl, heterocyclyl, oxyacyl, oxyacylamino, aminoacyloxy, haloalkyl, trihalomethyl, and the like.

Examples of particularly suitable optional substituents include F, C₁, Br, I, CH₃, CH₂CH₃, OH, OCH₃, CF₃, CH₂CF₃, OCF₃, NO₂, NH₂, COCH₃ and CN.

In certain aspects the present invention relate to compounds of Formula (I) or a pharmaceutically acceptable salt thereof:

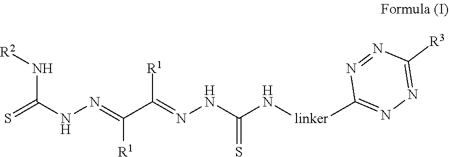

As described above, a requirement of a compound of Formula (I) is the ability to cross the blood brain barrier, with this ability influenced by the nature of the substituents at the variable R¹, R², R³ and linker positions. The present inventors have found that the following embodiments of Formula (I) with the specific options for each variable provide a compound with the requisite physical properties to ensure that the compounds are able to cross the blood brain barrier.

In an embodiment, R¹ is independently selected from an optionally substituted C₁-C₁₂ alkyl group. In an embodiment, each R¹ is an optionally substituted C₁-C₁₂ alkyl group. In an embodiment, each R¹ is an unsubstituted C₁-C₁₂ alkyl group. In an embodiment, each R¹ is an unsubstituted C₁ to C₃ alkyl group. In an embodiment, each R¹ is an unsubstituted C₁ alkyl group. In an embodiment, each R¹ is an unsubstituted C₂ alkyl group. In an embodiment, each R¹ is a methyl group. In an embodiment, each R¹ is an ethyl group. In embodiment, R¹ is a substituted C₁-C₁₂ alkyl group. In an embodiment, R¹ is a substituted C₁ to C₃ alkyl group. In an embodiment, R¹ is a C₁ to C₃ alkyl group substituted with one or more halogen groups. In an embodiment, R¹ is a C₁ to C₃ alkyl group substituted with one or more haloalkyl groups.

In an embodiment, R² is an optionally substituted C₁-C₁₂ alkyl group. In an embodiment, R² is an optionally substituted C₁-C₃ alkyl group. In an embodiment, R² is an unsubstituted C₁ to C₃ alkyl group. In an embodiment, R² is an unsubstituted C₁ alkyl group. In an embodiment, R² is an unsubstituted C₂ alkyl group. In an embodiment, R² is a methyl group. In an embodiment, R² is an ethyl group.

In an embodiment, R³ is an optionally substituted C₁ to C₁₂ alkyl group. In an embodiment, R³ is an unsubstituted C₁ to C₁₂ alkyl group. In an embodiment, R³ is an unsubstituted C₁ alkyl group. In an embodiment, R³ is a methyl group. In another embodiment, R³ is hydrogen.

In an embodiment, the linker is an optionally substituted alkylene group. In an embodiment, the linker is an optionally substituted phenylene group. In an embodiment, the linker is an optionally substituted benzylene group.

In an embodiment, the compound of Formula (I) has the following structure:

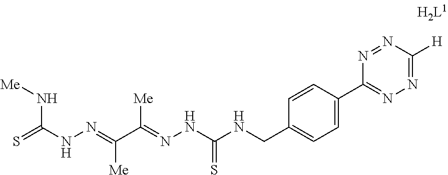 [SMILES: CNC(=S)N/N=C(C)/C(C)=N/NC(=S)NCc1ccc(-c2nncnn2)cc1]

where each of R¹ and R² are independently methyl, R³ is hydrogen and the linker is a benzylene group.

In another embodiment, the compound of Formula (I) has the following structure:

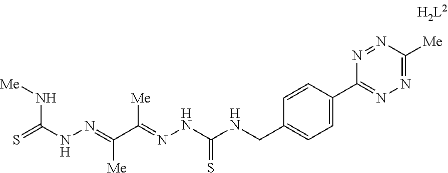 [SMILES: CNC(=S)N/N=C(C)/C(C)=N/NC(=S)NCc1ccc(-c2nnc(C)nn2)cc1]

where each of R¹, R² and R³ are independently methyl and the linker is a benzylene group.

In an embodiment, the compound of Formula (I) has the following structure:

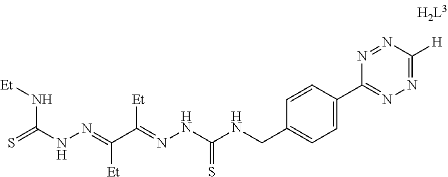 [SMILES: CCNC(=S)N/N=C(CC)/C(CC)=N/NC(=S)NCc1ccc(-c2nncnn2)cc1]

where each of R¹ and R² are independently ethyl, R³ is hydrogen and the linker is a benzylene group.

In an embodiment, the compound of Formula (I) has the following structure:

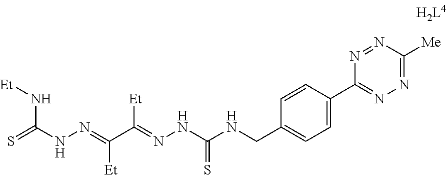 [SMILES: CCNC(=S)N/N=C(CC)/C(CC)=N/NC(=S)NCc1ccc(-c2nnc(C)nn2)cc1]

where each of R¹ and R² are independently ethyl, R³ is methyl and the linker is a benzylene group.

The present invention also relates to compounds of Formula (Ia), which is a complex of Formula (I) and a metal ion:

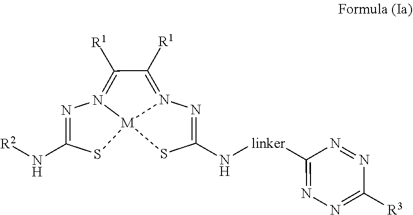

wherein M is a metal ion and R¹, R², R³ and the linker group are as defined above.

In an embodiment, the metal ion (M) of Formula (Ia) is an ion of a metal selected from the group consisting of Cu, Tc, Gd, Ga, In, Co, Re, Fe, Au, Ag, Rh, Pt, Bi, Cr, W, Ni, V, Ir, Zn, Cd, Mn, Ru, Pd, Hg and Ti. In a preferred embodiment, the metal ion of Formula (Ia) is a Cu ion.

The present inventors have found that the compounds of Formula (I) may be complexed with a copper ion. In an embodiment, a compound of Formula (I) is complexed with a copper ion.

In an embodiment, the copper ion is a copper(II) ion. In an embodiment, the copper ion is a Cu²⁺ ion. In some embodiments, the copper ion is a radioisotope of copper. In an embodiment, the copper ion is a ⁶⁴Cu ion. In another embodiment, the copper ion is a ⁶⁷Cu ion.

The present inventors have also found that the compounds of Formula (I) may also be complexed with other radioisotopes. In an embodiment, the radioisotope is a radioisotope of technetium (Tc). In an embodiment, the radioisotope is ^{99m}Tc. In an embodiment, the radioisotope is a radioisotope of gallium (Ga). In an embodiment, the radioisotope is ⁶⁸Ga. In an embodiment, the radioisotope is a radioisotope of cobalt (Co). In an embodiment, the radioisotope is ⁵⁵Co.

As described above, complexes of Formula (I) and a copper ion are tetradentate, with the sulfur atoms and two of the nitrogen atoms of Formula (I) chelating the copper ion. Embodiments of complexes of Formula (I) include:

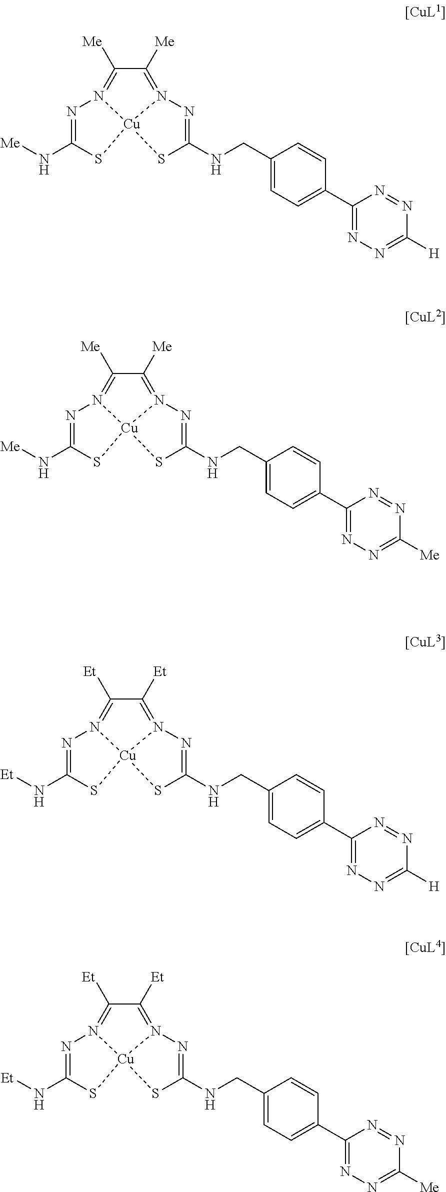

Embodiments of the above complexes where the copper ion is a radioisotope, for example a ⁶⁴Cu or a ⁶⁷Cu radioisotope, are also contemplated in the present invention.

The present invention also provides an antibody associated with a neurodegenerative disease or disorder, wherein the antibody is modified to include a click-receptive dienophile, wherein the click-receptive dienophile is bound to the antibody via a linker, wherein the linker comprises one or more ethylene glycol groups. In an embodiment, the click-receptive dienophile on the modified antibody is a trans-cyclooctene (TCO) group. In another embodiment, the click-reactive dienophile is bound to the antibody via a linking group. In an embodiment, the linking group that binds the click-reactive dienophile comprises one or more ethylene glycol groups. In an embodiment, the linking group that binds the click-reactive dienophile comprises a polyethylene glycol group.

The linker-dienophile group is installed on the surface of the antibody by reacting a suitable reagent with the antibody. For example, where the dienophile is a TCO functional group and the linker group is a polyethylene glycol moiety, a reagent bearing the TCO and linker group and a reactive centre can react with one or more amino acid side chains on the surface of the antibody. Examples of a reactive centre include various esters, aldehydes, carbodiimides, azides and the like. In an embodiment, the TCO and linker groups are bound to an ester. In an embodiment, the ester is an activated ester. In an embodiment, the activated ester is an NHS-ester, i.e. an N-hydroxysuccinimide ester. Given the known reactivity patterns of an activated ester, such as an NHS ester, the reagent containing the activated ester may react with one or more suitable amino acid side chains present on the surface of the antibody. For example, amino acids such a lysine, where the side chain contains a primary amine, contain a functional group that can react with an activated ester, such as an NHS ester. In an embodiment, the activated ester containing the TCO and linker group reacts with the side chain of a lysine group on the surface of the antibody.

In an embodiment, the antibody is modified by the installation of one or more TCO-polyethylene glycol (PEG) groups on the surface of the antibody. In an embodiment, the TCO-PEG group contains multiple PEG units, for example, the group installed on the surface of the antibody is a TCO-(PEG)ₙ-group, where n is an integer selected from 1, 2, 3, 4, 5, 6, 7, 8, 9 or 10. In a preferred embodiment, n is 4.

In another aspect, the present invention provides a composition comprising a compound of Formula (I), or a pharmaceutically acceptable salt thereof, together with one or more pharmaceutically acceptable excipients. The present invention also provides compositions comprising a modified antibody as discussed herein.

In an embodiment, the present invention provides compositions comprising a compound as described above together with one or more pharmaceutically acceptable excipients. Pharmaceutical compositions of this invention for parenteral injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres. The injectable formulations can be sterilized, for example, by filtration through a bacterial retaining filter, or by incorporating sterilizing agents in the form of sterile solid compositions that can be dissolved or dispersed in sterile water or other sterile injectable medium just prior to use.

The invention in other embodiments provides a pharmaceutical pack or kit comprising one or more containers filled with one or more of the ingredients of the pharmaceutical compositions of the invention. In such a pack or kit can be found at least one container having a unit dosage of a modified antibody as contemplated by the present invention and a compound of Formula (I) or a pharmaceutically acceptable salt thereof. Conveniently, in the kits, single dosages can be provided in sterile vials so that the clinician can employ the vials directly, where the vials will have the desired amount and concentration of a modified antibody, a compound of Formula (I) or a pharmaceutically acceptable salt thereof and a radioisotope which may be admixed with the compound of Formula (I) prior to use. Associated with such container(s) can be various written materials such as instructions for use, or a notice in the form prescribed by a governmental agency regulating the manufacture, use or sale of pharmaceuticals, imaging agents or biological products, which notice reflects approval by the agency of manufacture, use or sale for human administration.

The present invention provides a method for the in vivo diagnostic imaging of a neurodegenerative disease or disorder, the method comprising sequentially administering to a patient:
i) an antibody associated with the neurodegenerative disease or disorder, which has been modified to comprise a click-receptive dienophile; and
ii) a compound of Formula (I) or a pharmaceutically acceptable salt thereof as defined herein,

wherein the compound of Formula (I) is further complexed with a radioisotope and wherein the modified antibody covalently reacts in a click type reaction with the tetrazine of the compound of Formula (I) in situ.

The methods of the present invention involve the administration of a modified antibody containing a click-reactive dienophile and a compound of Formula (I) containing a tetrazine functional group for the imaging, diagnosis and/or treatment of a neurodegenerative disease or disorder.

A “click reaction” refers generally to a modular, high-yielding, stereospecific reaction that is insensitive to oxygen and water. An example of a click reaction is the Huisgen 1,3-dipolar cycloaddition of an alkyne to an azide. While this reaction often requires the use of a copper catalyst, other reactions that may be classified as a click reaction do not require the use of a metal catalyst. Another example of a click reaction is the reaction between a strained cyclooctene (or other strained alkene) with a tetrazine. The reaction between these two functional groups is fast, irreversible, requires equimolar amounts of each reactant and proceeds with high yields. The tetrazine initially reacts with the trans-cyclooctene in a [4+2] inverse electron demand Diels-Alder reaction to form a bridged intermediate. This is followed by a [4+2] retro Diels-Alder reaction accompanied with the release of nitrogen (N₂), an inert by product. Given the catalyst-free nature of the reaction between the cyclooctene and the tetrazine, this reaction is considered to be biorthogonal underpins the pre-targeting approach of the methods disclosed herein.

The modified antibody containing a click-reactive dienophile is administered first. The modified antibody may be administered regularly over a period of time, for example, daily over a period of one week, two weeks, three weeks or even four weeks. Once administered, the modified antibody exists in the circulatory system and due the physical properties of the modified antibody, crosses the blood brain barrier and enters the brain. Since the antibody (whether modified or unmodified) is specific for a particular protein, the antibody will preferentially accumulate in areas where the protein is of greater abundance, e.g. at locations in the brain containing protein aggregates. The accumulated antibody persists at sites in the brain where the targeted protein exists. Since the modified antibody may be administered at regular intervals over a period of time, gradual accumulation of the modified antibody can occur until the desired concentration of bound antibody in vivo is reached.

Once the modified antibody has reached the desired concentration in the brain, the administration of the modified antibody may be stopped in order to allow for any remaining antibody that is in the circulation and not bound to the target protein to be eliminated. This ensures that the modified antibody is located only where the target protein exists.

After the unbound portion of the modified antibody is cleared from the subject, the compound of Formula (I) as complexed with a radioisotope can be administered. In a similar manner, the radiolabelled compound of Formula (I) is allowed to circulate and given its physical characteristics, is able to cross the blood brain barrier and enter the brain. When a radiolabelled compound of Formula (I) encounters the modified antibody, the tetrazine of the compound of Formula (I) reacts with the click-reactive dienophile that was previously installed on the surface of the antibody. Since the kinetics of the click-type reaction between the tetrazine and the dienophile is highly favoured, the reaction proceeds quickly with the formation of new covalent bonds. The formation of these new covalent bonds connects the radiolabelled compound of Formula (I) to the site in the brain at which the protein associated with the neurodegenerative disease or disorder is located, via the modified antibody. Given the strength of the new covalent bonds that are formed, the radiolabelled compound of Formula (I) is retained at the desired site. Since the compound of Formula (I) is complexed with a radioisotope, the decay pattern of the radioisotope may be detected by appropriate means, for example by PET or SPECT imaging. The specific radioisotope is selected based on the half-life of the radioisotope and also the manner in which imaging is intended. In an embodiment, the radioisotope is a copper radioisotope, preferably a ⁶⁴Cu or a ⁶⁷Cu radioisotope. These radioisotopes of copper have a half-life that is significantly longer, for example, ⁶⁴Cu has a half-life of 12.7 hours, which results in a greater radiochemical yield. The longer half-life in conjunction with the specific decay characteristics of the copper radioisotope, allows images to be collected over a longer period of time. Since this copper radioisotope allows for imaging to be performed over a longer time, the images obtained are typically of higher quality and of greater contrast. This allows for greater certainty when interpreting images and reaching a diagnosis of a neurodegenerative disease or disorder. This is in contrast to known radioisotopes such as ¹¹C and ¹⁸F, where the half-life is considerably shorter, which limits the usefulness of these isotopes in diagnostic imaging and therapy.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the disclosure.

### EXAMPLES

The following examples are illustrative of the disclosure and should not be construed as limiting in any way the general nature of the disclosure of the description throughout this specification.

The compounds of the various embodiments may be prepared using the reaction routes and synthesis schemes as described below, employing the techniques available in the art using starting materials that are readily available. The preparation of particular compounds of the embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g. by appropriately protecting interfering groups, by changing to other suitable reagents known in the art, or by making routine modifications of reaction conditions. A list of suitable protecting groups in organic synthesis can be found in T. W. Greene's Protective Groups in Organic Synthesis, 3rd Edition, John Wiley & Sons, 1991. Alternatively, other reactions disclosed herein or known in the art will be recognized as having applicability for preparing other compounds of the various embodiments.

General Experimental Details

All solvents and reagents were purchased from standard commercial suppliers and were used as received. 64CuCl₂ solution (0.02 M HCl, no carrier added) was produced at Sir Charles Gairdner Hospital (Nedlands, Western Australia) through the ⁶⁴Ni(p,n)⁶⁴Cu nuclear reaction.

¹H, ¹³C, COSY, HSQC, HMBC were all recorded using a Varian FT-NMR 400 of FT NMR 500 spectrometer (Varian, Calif., USA). All ¹H NMR spectra were acquired at 400 MHz or 500 MHz and ¹³C spectra were acquired at 101 MHz or 126 MHz. The reported peaks were all referenced to solvent peaks in the order of parts per million at 25° C.

ESI-QTOF MS was collected on an Exactive Plus Orbitrap Infusion mass spectrometer (Exactive Series, 2.8 Build 268801, ThermoFisher Scientific). Analysis was performed using Xcalibur 4.0.27.10 (ThermoFisher Scientific). Protein samples were analysed on Agilent 6220 ESI-TOF LC/MS Mass Spectrometer coupled to an Agilent 1200 LC system (Agilent, Palo Alto, Calif.). All data were acquired, and reference mass corrected via a dual-spray electrospray ionisation (ESI) source. Acquisition was performed using the Agilent Mass Hunter Acquisition software version B.02.01 (B2116.30). Ionisation mode: Electrospray Ionisation; Drying gas flow: 7 L/min; Nebuliser: 35 psi; Drying gas temperature: 325° C.; Capillary Voltage (Vcap): 4000 V; Fragmentor: 300 V; Skimmer: 65 V; OCT RFV: 250 V; Scan range acquired: 300-3200 m/z Internal Reference ions: Positive Ion Mode=m/z=121.050873 & 922.009798.

Protein desalting and chromatographic separation was performed using an Agilent Poroshell C18 2.1×75 mm, 5 μm column using 5% (v/v) acetonitrile ported to waste (0-5 min). Upon desalting of the sample, the flow was ported back into the ESI source for subsequent gradient elution with (5% (v/v) to 100% (v/v)) acetonitrile/0.1% formic acid over 8 min at 0.25 mL/min. Analysis was performed using Mass Hunter version B.06.00 with BioConfirm software using the maximum entropy protein deconvolution algorithm; mass step 1 Da; Baseline factor 3.00; peak width set to uncertainty.

Non-radioactive analytical HPLC were performed on Agilent 1200 series HPLC system fitted with an Alltech Hypersil BDS-C18 (4.6×150 nm, Sum) with a 1 mL/min flow rate; system A: gradient elution of Buffer A=0.1% TFA in H₂O and Buffer B=0.1% TFA in acetonitrile (0 to 100% B in A over 25 min) and UV detection at λ 220, 254, and 350 nm. Several chromatographic systems were used for purification steps.

Semipreparative RP-HPLC (Agilent 1200 series HPLC system on a Lunar C18 column, 100 Å 21.2×250 mm, 5 μm) with an 8 mL/min flow rate. System B: gradient elution of Buffer A=0.1% TFA in H₂O and Buffer B=0.1% TFA in acetonitrile (0 to 40% B in A at 30 min, 40-100% B in A at 35 min, 100% B at 40 min) and detection at 214 and 254 nm. System C: System B with the following gradient (0 to 100% B in A at 40 min, 100% B at 44 min, 100 to 0% B in A at 45 min).

Radioactive analytical HPLC was performed on a Shimadzu SCL-10A VP/LC-10 AT VP system with a Shimadzu SPD-10A VP UV detector followed by a radiation detector (Ortec model 276 photomultiplier base with preamplifier, Ortec 925-SCINT ACE mate preamplifier, BIAS supple and SCA, Bicron 1M 11/2 photomultiplier tube). Column: Phenomenex Luna 5 μm C18(2) 150×4.6 m 100 A. Gradient elution of Buffer A=0.05% TFA in H₂O and Buffer B=0.05% TFA in acetonitrile (50 to 100% B in A at 17 min, 100% at 21 min and 50% at 23 min) and detection at, 254, and 350 nm. Radio-iTLC were analysed using a Raytest Rita-Star TLC scanner.

Electrochemistry was conducted using an AUTOLAB PGSTAT100 with GPES V4.9 software. A glassy carbon working electrode, a Pt/Ti wire counter electrode and a leakless miniature Ag/AgCl reference electrode were used. Ferrocene was used as an internal reference (E^{o′}(Fc/Fc+)=0).

Microanalysis measurements were carried out by The Campbell Microanalytical Laboratory in the Department of Chemistry, University of Otago, Union Place, Dunedin, New Zealand.

X-ray crystallography of CuL³ was mounted in low temperature oil then cooled to 130 K using an Oxford low temperature device. Intensity data were collected at 130 K with an Oxford XCalibur X-ray diffractometer (CuL³) with Sapphire CCD detector using Cu Kα radiation (graphite crystal monochromators λ=1.54184 Å). Data were reduced and corrected for absorption. The structures were solved by direct methods and difference Fourier synthesis using the SHELX suite of programs as implemented within the WINGX2 3.38 software.

Thermal ellipsoid plots were generated using the programs ORTEP-3 integrated with the SHELX suite of programs.

SEC-ICP-MS was performed using HPLC (model 1200, Agilent). Samples were chromatographically separated using a Superdex 200 (5×150 mm) with 200 mM ammonium nitrate containing internal standard (133Cs, 121Sb; 10 μg L⁻¹ each), pH 7.5, at a flow rate of 0.4 ml/min. The HPLC was directly connected to MicroMist nebulizer (Glass Expansion, Australia) fitted to an Agilent Technologies 203 7700x ICP-MS. Helium was used as the collision gas (3 mL min⁻¹) to minimize polyatomic interferences with all elements. The following elements were analyzed: ²³Na, ²⁴Mg, ³⁹K, ⁴⁴Ca, ⁵²Cr, ⁵⁵Mn, ⁵⁶Fe, ⁵⁹Co, ⁶⁰Ni, ⁶³Cu, ⁶⁶Zn, ⁸⁵Rb, ⁹⁵Mo, ¹¹¹Cd, ¹²¹Sb, ¹³³Cs and ¹⁸²W.

Brain tissues isolated from the frontal cortex of AD and healthy control subjects were fixed in 10% formalin/PBS and embedded in paraffin. Serial sections (7 μm) were deparaffinized and treated with 80% formic acid (5 min) and endogenous peroxidase activity was blocked utilizing 3% hydrogen peroxide. The section was then treated with blocking buffer (20% fetal calf serum, 50 mM Tris-HCl, 175 mM NaCl pH 7.4) before incubation with primary antibody to AO (1E8; 1:500, 6E10; 1:1500) for one hour at room temperature. Visualization of antibody reactivity was achieved using the LSABTM+ kit (DAKO) and hydrogen-peroxidase-diaminobenzidine (H₂O₂-DAB) to visualise the Aβ deposits. Bright field images were visualized using a Leica DMIL LED microscope.

The copper-64 complexes were prepared by diluting [⁶⁴Cu]CuCl₂ (aq) (pH 1, 38 μL, 50 MBq) with aqueous sodium acetate (0.1 M, pH 5.5 100 μL) to pH 5, then H₂L³ in DMSO was added (1 mg/mL, 5 μL) at room temperature for 35 minutes. 30 μL of the reaction solution was taken for analysis by reverse phase radio-HPLC. To test the reactivity of the tetrazine functional group of [⁶⁴Cu]CuL³ and ensure no breakdown of the tetrazine ring due to radiolysis, a solution of [⁶⁴Cu]CuL³ (10 MBq) in ethanol (10%), DMSO (8%), aqueous sodium acetate (0.1 M, pH 5.5) was prepared. TCO-OH (3 μL, 3.2 mg/mL, 6 equivalents) was added to the solution and reacted for 10 minutes before 40 μL of the reaction solution was taken for analysis by reverse phase radio-HPLC. For dynamic mouse imaging studies, [CuL³] reaction mixture (25 MBq, 70 uL) was prepared with a (final concentration of DMSO (8%), ethanol (10%) in NaOAc buffer, 3 MBq activity in 150 uL dose per mouse). Wild type (WT) mice (n=3) underwent microPET/computed tomography (CT) imaging (GENISYS 8 PET/CT, Sofie Biosciences). Mice were anesthetized under 2% isoflurane, placed in a heated G8 imaging chamber and catheterized with [⁶⁴Cu][CuL³] by the tail vein. The mice were injected as the PET scan was started, with a 1.5 min CT at the end of the 30 min PET acquisition. The list-mode data-framing sequence for the 30 min scan was 15×1 s, 15×15 s, 26×60 s for image display and 15×15 s, 26×60 s for quantitation. Image display and analysis carried out with VivoQuant (version 3.0). 3D region of interests (ROIs) have been drawn around the brain, harderian glands, heart, lungs, liver, kidneys and background to generate time-activity curves (TACs) in units of standardised uptake values (SUV).

### Example 1—Synthesis of Compounds of Formula (I) and Complexes Thereof

The bis(thiosemicarbazone) ligands of Formula (I) are synthesised by condensation reactions between either 2,3-butanedione or 3,4-hexanedione (depending on the desired degree of alkylation on the ligand backbone) and 4-substituted-3-thiosemicarbazides. Non-symmetrical bis(thiosemicarbazone) ligands where one limb bears a 4,4′-dimethylthiosemicarbazone limb react with primary amines to give products where an amine functional group is incorporated into the ligand (see Scheme 1).

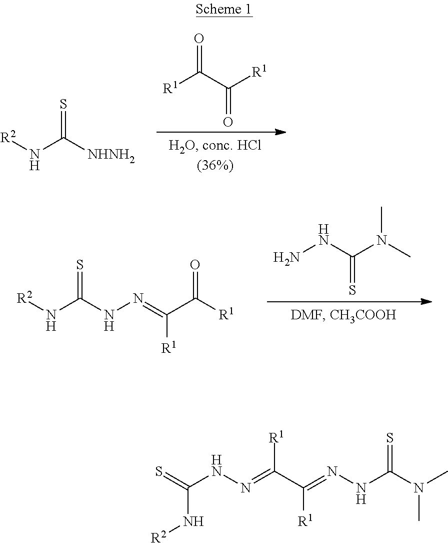

Due to the reactivity of the tetrazine moiety, traditional trans-amination reactions that require long heating times at elevated temperatures were not suitable. Instead, utilizing microwave radiation to facilitate shorter reaction times was used. These compounds were purified by silica chromatography or recrystallisation and characterized by NMR spectroscopy, high resolution mass spectrometry, HPLC and microanalysis. Copper complexes {CuL¹⁻⁴} were formed by addition of copper(II) acetate hydrate to mixtures of the ligands in DMF (H₂L¹ and H₂L²) or methanol (H₂L³ and H₂L⁴). Acetate ions deprotonate the hydrazinic protons on the bis(thiosemicarbazone) backbone and the ligand acts as a dianionic tetradentate N₂S₂ donor forming charge neutral complexes (see Scheme 2).

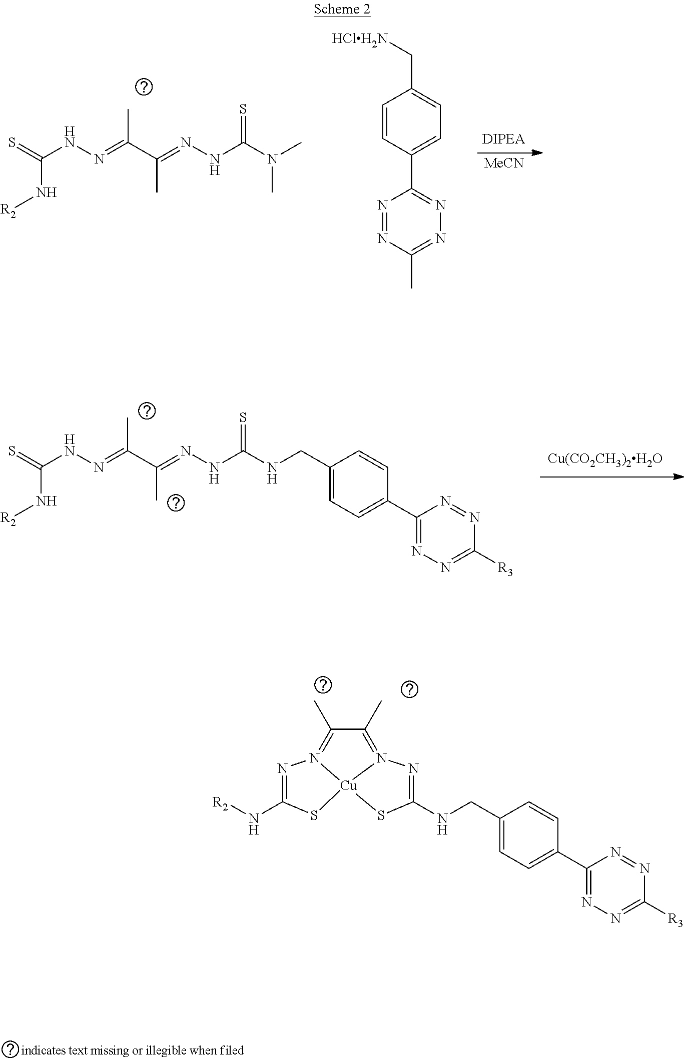

Dissymmetric bis(thiosemicarbazone) compounds containing a dimethylamino functional group were prepared according to a literature procedure (see Paterson, B. M.; Karas, J. A.; Scanlon, D. B.; White, J. M.; Donnelly, P. S., Inorg Chem 2010, 49, 1884-93).

Bis(thiosemicarbazone) compounds, 4-(1,2,4,5-tetrazin-3-yl)phenyl) methanamine hydrochloride (see Maggi, A.; Ruivo, E.; Fissers, J.; Vangestel, C.; Chatterjee, S.; Joossens, J.; Sobott, F.; Staelens, S.; Stroobants, S.; Van Der Veken, P.; Wyffels, L.; Augustyns, K., Org Biomol Chem 2016, 14, 7544-51) or (4-(6-methyl-1,2,4,5-tetrazin-3-yl)phenyl)methanamine hydrochloride (see Denk, C.; Svatunek, D.; Filip, T.; Wanek, T.; Lumpi, D.; Frohlich, J.; Kuntner, C.; Mikula, H., Angew Chem Int Ed Engl 2014, 53, 9655-9) in acetonitrile were irradiated with microwave radiation for 5 minutes at 90° C. The crude organic mixtures were then subjected to one of two general workups. Workup A: upon cooling a bright pink precipitate formed, H₂O (2-3 mL) was added to aid precipitation the reaction suspension was centrifuged, washed with H₂O (2 mL), cold ethanol (2 mL) and diethyl ether then dried in vacuo to yield a pink powder. Workup B: upon cooling, the reaction mixture was taken to dryness under reduced pressure. The precipitate was purified via column chromatography (SiO₂, 10-40% EtOAc in CH₂Cl₂), fractions were collected and taken to dryness in vacuo to yield a bright pink/purple solid.

### Dimethyl-4-((4-(1,2,4,5-tetrazin-3-yl)phenyl)methanamine)-4′-methylbis(thiosemicarbazone), H₂L¹

Dimethyl-4′-methylbis(thiosemicarbazone) (22.0 mg, 0.08 mmol), 4-(1,2,4,5-tetrazin-3-yl)phenyl) methanamine hydrochloride (22.1 mg, 0.096 mmol) and triethyl amine (13 μL, 0.096 mmol) were suspended in acetonitrile (1.5 mL) and irradiated with μW radiation then subjected to workup A (21.2 mg, 0.051 mmol, 64%). Rₜ: 11.39 min (system A). ESI MS {M+H}⁺: 417.1392 calculated for (C₁₆H₂₀N₁₀S₂)⁺: 417.1387. ¹H NMR (500 MHz, DMSO-d₆) δ 10.58 (s, 1H), 10.43 (s, 1H), 10.22 (s, 1H), 9.06 (s, 1H), 8.48 (d, J=8.4 Hz, 2H), 8.39 (d, =4.6 Hz, 1H), 7.60 (d, J=8.5 Hz, 2H), 4.98 (d, J=6.3 Hz, 2H), 3.03 (d, J=4.6 Hz, 3H), 2.25 (d, J=9.5 Hz, 6H), 2.08 (s, 1H). ¹³C NMR (126 MHz, DMSO) δ 178.5, 178.3, 165.3, 157.9, 148.6, 147.7, 144.5, 130.1, 127.8, 127.6, 46.6, 31.0, 11.7, 11.6. Measured: C: 44.9%, H: 4.9%, N: 31.1% calculated C: 45.0%, H: 5.2%, N: 31.2% (0.8 H₂O. 0.2 EtOAc)

### Dimethyl-4-((4-(1,2,4,5-tetrazin-3-yl)phenyl)methanamine)-4′-methylbis(thiosemicarbazonato)-copper(II), [Cu^{II}L¹]

H₂L¹ (4.78 mg, 0.011 mmol) and Cu(II)OAc.H₂O (2.59, 0.013 mmol) were dissolved in DMF (0.5 mL), before the addition of H₂O. The precipitate was collected via centrifugation, and the solid was washed with H₂O, ethanol, and diethyl ether, then taken to dryness in vacuo to yield a dark red solid. (4.62 mg, 0.009 mmol, 88%). Rₜ: 11.521 mins (system A). ESI MS {M+H}⁺: 478.0539 calculated for (C₁₆H₁₈CuN₁₀S₂)⁺: 478.0526. Measured: C 42.5%, H 4.7% N 22.2%: calculated C 40.2%, 3.8%, N 29.3%.

### Dimethyl-4-((4-(6-methyl-1,2,4,5-tetrazin-3-yl)phenyl)methanamine)-4′-methyl bis(thiosemicarbazone), H₂L²

Dimethyl-4′-methylbis(thiosemicarbazone) (20.0 mg, 0.073 mmol), (4-(6-methyl-1,2,4,5-tetrazin-3-yl)phenyl)methanamine hydrochloride (20.7 mg, 0.087 mmol) and triethyl amine (12 μL, 0.087 mmol) were suspended in acetonitrile (1.5 mL) and irradiated with μW radiation then subjected to workup A (18.5 mg, 0.043 mmol, 59%). Rₜ: 11.39 min (system A). ESI MS {M+H}⁺: 431.1545 calculated for (C₁₇H₂₂N₁₀S₂)⁺: 431.1543. ¹H NMR (500 MHz, DMSO-d₆) δ 10.43 (s, 1H), 10.22 (s, 1H), 9.05 (t, J=6.3 Hz, 1H), 8.44 (d, J=8.4 Hz, 2H), 8.39 (d, J=4.6 Hz, 1H), 7.59 (d, J=8.5 Hz, 2H), 4.98 (d, J=6.2 Hz, 2H), 3.03 (d, J=4.6 Hz, 3H), 2.99 (s, 3H), 2.26 (s, 3H), 2.24 (s, 3H). ¹³C NMR (126 MHz, DMSO) δ 178.6, 178.5, 167.0, 163.2, 148.7, 147.9, 144.1, 130.3, 128.0, 127.3, 46.8, 39.5, 31.2, 20.8, 20.8, 11.8, 11.7. Measured C: 47.4%, H: 5.1% N: 32.0% calculated C: 47.2% H: 5.2% N: 32.4% (0.1 H₂O).

### Dimethyl-4-((4-(6-methyl-1,2,4,5-tetrazin-3-yl)phenyl)methanamine)-4′-methylbis(thiosemicarbazonato)-copper(II), [Cu^{II}L²]

Following the same procedure employed for the synthesis of [Cu^{II}L¹], H₂L² (13.5 mg, 0.031 mmol) and Cu(II)OAc.H₂O (6.84 mg, 0.034 mmol) were used to prepare [Cu^{II}L¹] yielding a dark red solid (12.97 mg, 0.026 mmol, 85%). Rₜ: 12.121 mins (system A). ESI MS {M+H}⁺: 492.0678 calculated for (C₁₇H₂₀CuN₁₀S₂)⁺: 492.0683. Measured: C 40.6%, H: 4.1% N: 27.2%: calculated C: 40.2%, H: 4.3%, N: 27.6% (1.H₂O)

### Diethyl-4-((4-(1,2,4,5-tetrazin-3-yl)phenyl)methanamine)-4′-ethylbis(thiosemicarbazone), H₂L³

Diethyl-4′-methylbis(thiosemicarbazone) (6.44 mg, 0.020 mmol), 4-(1,2,4,5-tetrazin-3-yl)phenyl) methanamine hydrochloride (5.01 mg, 0.022 mmol) and DIPEA (3 μL, 0.023 mmol) were suspended in acetonitrile (1.5 mL) and irradiated with μW radiation then subjected to workup B (6.08 mg, 0.013 mmol, 67%). Rₜ: 13.31 min (system A). ESI MS {M+H}⁺: 459.1853 calculated for (C₁₉H₂₇N₁₀S₂)⁺: 459.1856. ¹H NMR (500 MHz, DMSO-d₆) δ10.64 (s, 1H), 10.58 (s, 1H), 10.35 (s, 1H), 8.98 (t, J=6.2 Hz, 1H), 8.48 (d, J=8.3 Hz, 2H), 8.33 (t, J=5.8 Hz, 1H), 7.60 (d, J=8.3 Hz, 2H), 4.99 (d, J=6.1 Hz, 2H), 3.63-3.57 (m, 2H), 2.93 (q, J=7.3 Hz, 4H), 1.14 (t, J=7.1 Hz, 3H), 0.92 (dt, J=14.4, 7.4 Hz, 6H). ¹³C NMR (126 MHz, DMSO) δ 179.1, 177.9, 165.9, 158.6, 152.0, 151.3, 145.1, 130.8, 128.4, 128.2, 47.2, 39.0, 17.6, 17.4, 14.8, 11.4, 11.3.

### Diethyl-4-((4-(1,2,4,5-tetrazin-3-yl)phenyl)methanamine)-4′-ethylbis(thiosemicarbazonato)-copper(H), [Cu^{II}L³]

H₂L³ (15.67 mg, 0.034 mmol) and Cu(II)OAc.H₂O (8.37 mg, 0.042 mmol) were suspended in Methanol and allowed to stir at room temperature overnight. The suspension was centrifuged and washed with H₂O and ethanol then taken to dryness in vacuo. The residue was purified via column chromatography (SiO₂, 0-2% DCM in CH₂Cl₂) to yield a red solid. (15.89 mg, 0.030 mmol, 88%). Rₜ: 14.151 min (system A). ESI MS {M+H}⁺: 520.0991 calculated for (C₁₉H₂₄CuN₁₀S₂)⁺: 520.0996.

### Diethyl-4-((4-(6-methyl-1,2,4,5-tetrazin-3-yl)phenyl)methanamine)-4′-ethylbis(thiosemicarbazone), H₂L⁴

Diethyl-4′-methylbis(thiosemicarbazone) (12.1 mg, 0.07 mmol), (4-(6-methyl-1,2,4,5-tetrazin-3-yl)phenyl)methanamine hydrochloride (20.0 mg, 0.085 mmol) and DIPEA (154, 0.085 mmol) were added to acetonitrile (1.5 mL), then irradiated with μW radiation then subjected to workup B (22.14 mg, 0.046 mmol, 66%). Rₜ: 13.655 min. ESI MS {M+H}⁺: 473.2016 calculated for (C₂₀H₂₉N₁₀S₂)⁺: 473.2013. ¹H NMR (500 MHz, DMSO-d₆) δ 10.56 (s, 1H), 10.28 (s, 1H), 8.95 (t, J=6.1 Hz, 1H), 8.42 (d, J=8.2 Hz, 2H), 8.31 (t, J=5.8 Hz, 1H), 7.57 (d, J=8.2 Hz, 2H), 4.97 (d, J=6.0 Hz, 2H), 3.62-3.56 (m, 2H), 2.98 (s, 3H), 2.95-2.83 (m, 4H), 1.13 (t, J=7.1 Hz, 3H), 0.91 (dt, J=14.3, 7.4 Hz, 6H). ¹³C NMR (126 MHz, DMSO) δ 179.1, 177.9, 167.5, 163.7, 152.0, 151.4, 144.5, 130.8, 128.4, 127.8, 47.2, 39.1, 21.3, 17.6, 17.5, 14.8, 11.4, 11.3. Measured C: 51.4% H: 6.3%, N: 27.4% calculated C: 51.1%, H: 6.2%, N: 27.1% (0.5 EtOAc).

### Diethyl-4-((4-(6-methyl-1,2,4,5-tetrazin-3-yl)phenyl)methanamine)-4′-ethylbis(thiosemicarbazonato)-copper(II), [Cu^{II}L⁴]

Following the same procedure employed for the synthesis of [Cu^{II}L¹], H₂L⁴ (22.14 mg, 0.047 mmol) and Cu(II)OAc.H₂O (13.18 mg, 0.056 mmol). were used to prepare [Cu^{II}L⁴]. Solvent volume was reduced in vacuo and the residue was purified via column chromatography to yield a deep purple solid (12.13 mg, 0.023 mmol, 48%). ESI MS {M+H}⁺: 534.1158 calculated for (C₂₀H₂₆CuN₁₀S₂)⁺: 534.1152. Rₜ: 14.52 min (system A). Measured: C 44.1%, H: 4.9%, N: 24.1%: calculated: C: 44.0%, H: 5.3%, N: 24.2% (1 H₂O.0.3 EtOAc).

### Example 2—Radiolabelling of [CuL³]

H₂L³ was radiolabelled with [⁶⁴Cu]CuCl₂ in sodium acetate buffer (0.1 M, pH 5.5) for 35 minutes at room temperature (20-25° C.) and purity of the radiolabelled complex was determined via RP-HPLC (see FIG. 5). A single peak in the non-radioactive complex (λ=350 nm) corresponds to the radioactive tracer (scintillation) at 13.7 and 13.9 minutes, respectively; the data shows no free copper was present in the sample, with the complexation performed under relatively mild conditions.

### Example 3—Crystallisation and Crystallographic Analysis of [CuL³]

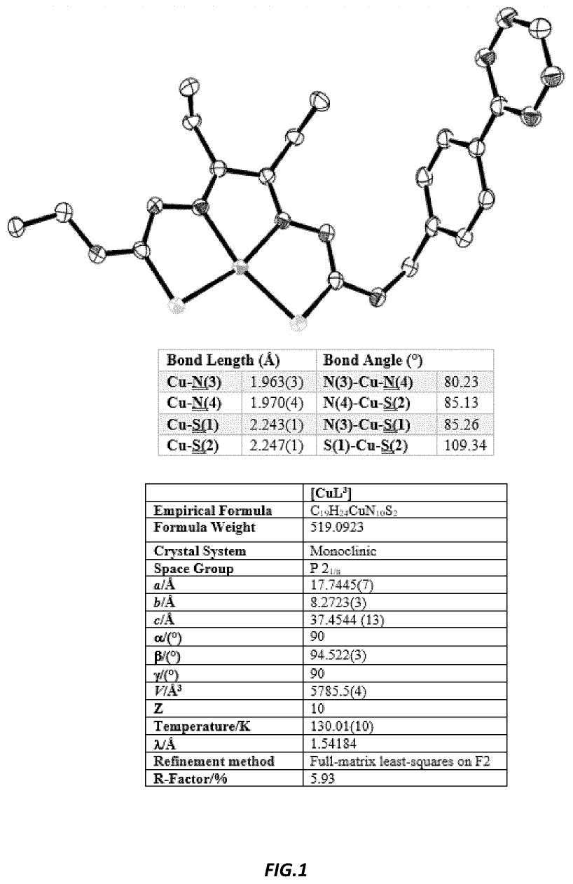

Crystallisation of [CuL³] from slow evaporation of a DMSO solution produced crystals of suitable quality for X-ray diffraction analysis (FIG. 1). The Cu^{II} is in a CuN₂S₂ square planar environment. The hydrazinic nitrogen atoms deprotonate upon complexation, resulting in bond delocalisation and a lengthening of the C—S bonds, resulting in the Cu—N bonds being shorter than the Cu—S bonds. The angle of the side chain, defined by the atoms C(10)-N(6)-C(11) is 126.6°; however, this angle is strongly determined by the crystal packing, hydrogen bonding to adjacent molecules and to the solvent molecule, DMSO (omitted from the crystal structure for clarity). The four-coordinate copper atom sits 0.06 Å out of the plane of the N₂S₂ square planar donor system with the distortion from ideal square planar geometry highlighted by the bond angle S(1)-Cu—S(2) of 109.34°.

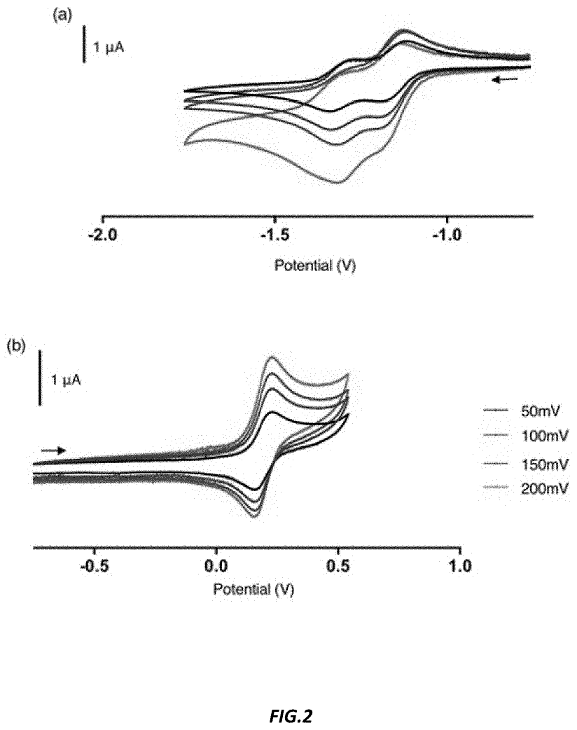

### Example 4—Cyclic Voltammetry of Free Ligands and Complexes

[CuL³] undergoes a quasi-reversible reduction at E^{o′}=−1.15 V which is tentatively assigned to a Cu^{II}/Cu^{I} redox process similar to the parent compound [Cu^{II}(atsm)].¹⁸⁻¹⁹ The secondary redox process at E^{o′}=−1.31 V is tentatively assigned to a ligand-based process, indicating a reduction of the tetrazine ring functional group (see FIG. 2). This assumption is supported by the cyclic voltammetry of the free ligands, H₂L^{3/4} (vide infra). There is an oxidation process occurring at E^{o′}=0.19 V for [CuL³], with a peak separation of 64 mV. Under the same conditions, the Fc/Fc⁺ couple displays a peak separation of 67 mV, suggesting these processes are both one-electron transfer systems.

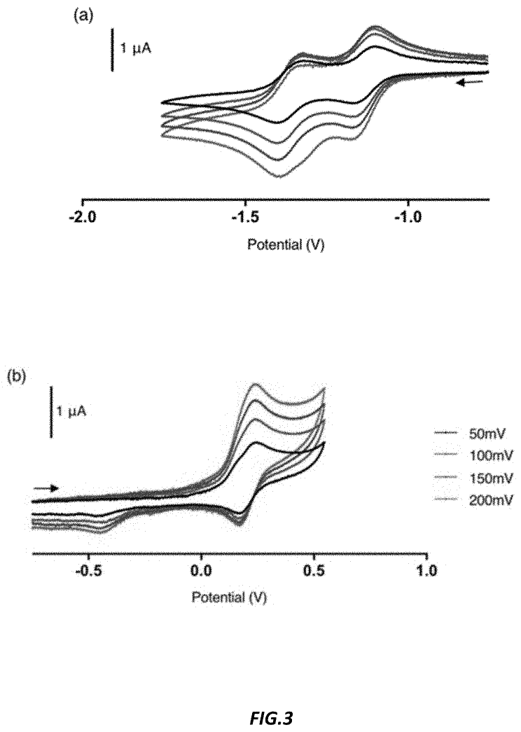

Similarly, in [CuL⁴], there were two processes occurring (see FIG. 3). The Cu^{II}/Cu^{I} redox couple is tentatively assigned at the reduction potential at E^{o′}=−1.12 V, with a ligand-based process at E^{o′}=−1.37 V There is an oxidation process occurring at E^{o′}=0.21 V, which can tentatively be assigned as a Cu^{II}/Cu^{III} redox process and showed substantially reduced current on the return sweep (reduction), resulting in an I_{pc}/Iₚₐ ratio of 0.39.

The electrochemistry of H₂L³ and H₂L⁴ was investigated to further elucidate whether one of the two reduction processes was ligand-based. Tetrazines that are substituted by heteroatoms or aromatic ring structures can be reversibly reduced in organic solvents due to their electron deficient character. They are able to accept one electron to give an anion radical (which is stable in the absence of acids). The differences in E° values for the reversible reduction to corresponding anion radical reflects the electronic influence of their substituents, which is consistent with the electron withdrawing character of the substituents at the 3- and 6-position.

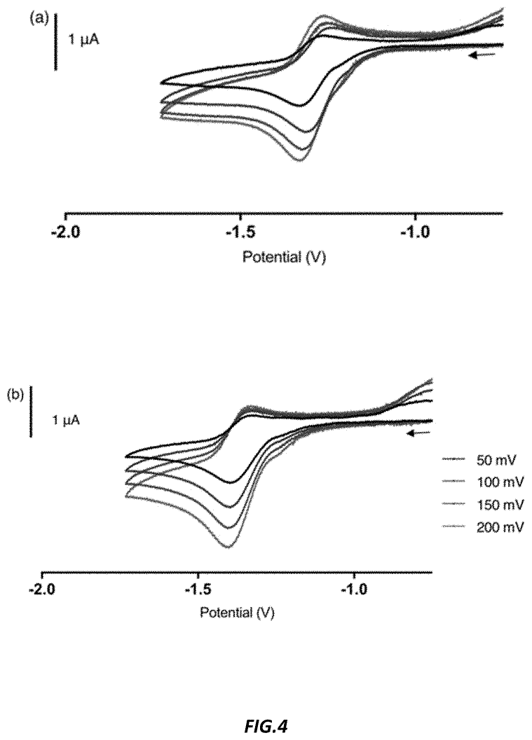

H₂L³ and H₂L⁴ were shown to have reduction potentials of E^{o′}=−1.29 V and E^{o′}=−1.37 V, respectively (see FIG. 4). No electrochemical processes were observed for either H₂L³ or H₂L⁴ at positive potentials (oxidation) in the voltammogram. These potentials correlate well with the electron deficiency of the substituent at the 6-position of the tetrazine, as H₂L³ has a higher reduction potential and is more readily reduced. Comparison of the cyclic voltammograms of the ligands and complexes makes it possible to tentatively assign the reduction processes at E^{o′}=−1.31 V ([CuL³]) and E^{o′}=−1.37 V ([CuL⁴]) to ligand-based processes due to the reduction of the tetrazine ring.

### Example 5—Surface Modification of Proteins/Antibodies

TCO-PEG4-NHS is an activated ester used for the modification of surface lysine resides in proteins or antibodies via covalent bonds. To ensure the reactivity of TCO is maintained after antibody modification it is necessary to use TCO conjugated to a small poly(ethylene glycol) (PEG), which is a hydrophilic linker, since the TCO-NHS ester is unsuitable for protein modification. The addition of a PEG linker also increases solubility of the TCO ring and prevents steric hindrance of the bulky cycloaddition reaction near the antibody surface. It also inhibits interaction of the hydrophobic TCO with external domains of the antibody or burying within the interior domains to avoid aqueous solvent. Herceptin (HER) is a clinically approved monoclonal antibody with a molecular weight of 148 kDa. Herceptin was available in relatively large quantities (>0.5 mg) and was used for proof of concept studies.

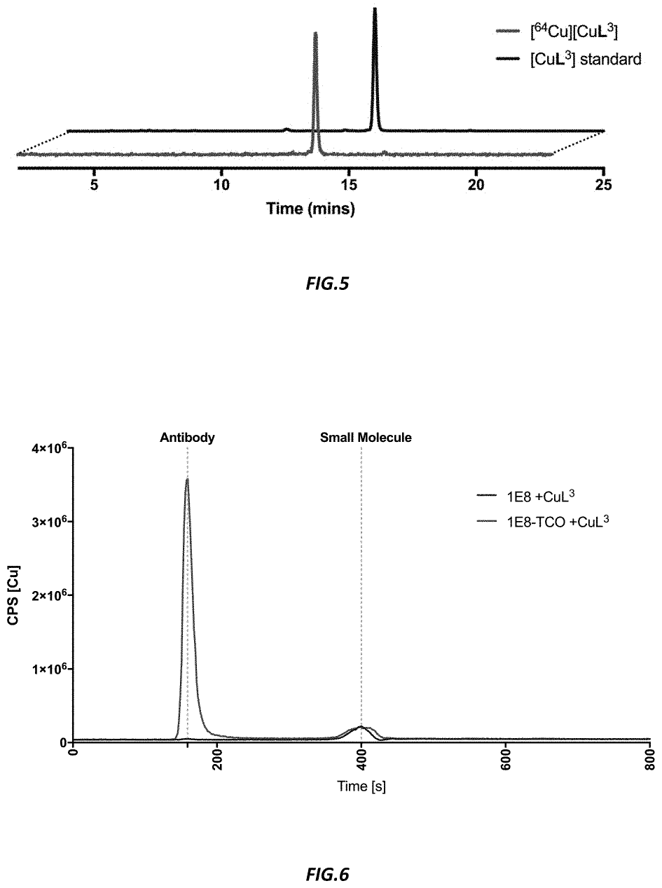

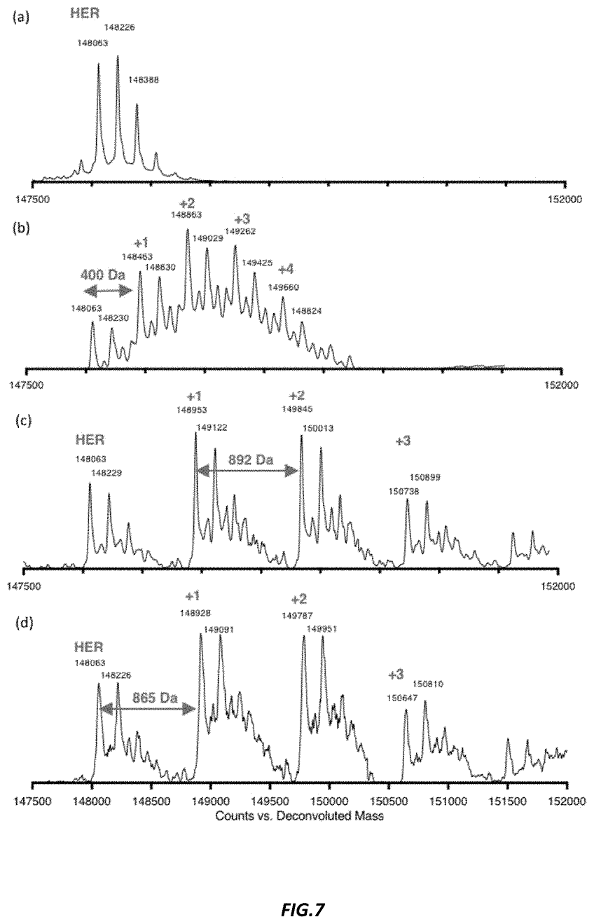

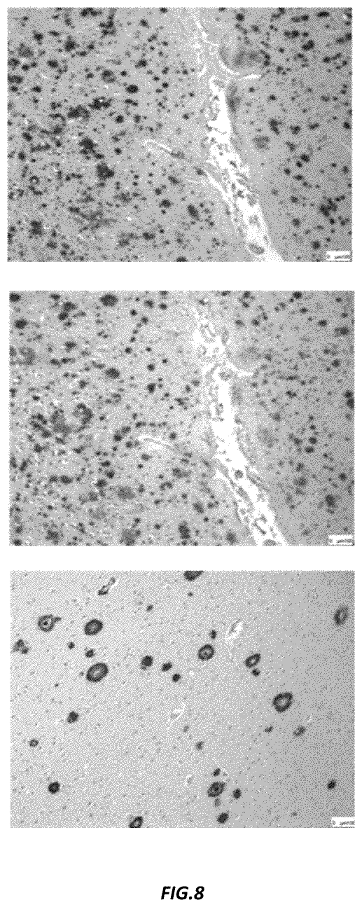

The conjugation reaction was followed via ESI-TOF MS to determine the number of TCO moieties covalently attached to HER. The MS of unmodified Herceptin (see FIG. 6a) shows three peaks, which correlate with different degrees of glycosylation branching on the antibody surface. Incubation of 10 equivalents of TCO-PEG4-NHS ester for 1 hour resulted in the attachment of between 1 and 4 TCO-PEG4 fragments per antibody (see FIG. 6b). Further increasing the number of TCO-PEG4 attachments per antibody may interfere with the antigen binding site or alter the pharmacokinetic properties of the antibody. Excess TCO-PEG4-NHS reagent was removed via spin filtration. The reactivity of the TCO functional groups on HER-PEG4-TCO was confirmed with the addition of 10 equivalents of either [CuL²] or [CuL³]. It is possible to follow the reaction via the change in mass between peaks of highest intensity in the deconvoluted MS. Before the ‘click’ reaction, the peaks were separated by 400 Da, which is equivalent to the reaction between TCO-PEG4-NHS in the formation of a covalent amide bond to surface lysine residues. For [CuL³] after the ‘click’ reaction, each peak corresponds to a mass difference of 892 Da, while for [CuL²] mass difference is 865 Da (see FIGS. 6c and 6d), which shows the covalent reaction between the tetrazine complexes and TCO moieties on the antibody surface. Although [CuL²] exhibits poor solubility in aqueous buffers, it is possible to see complete reaction with TCO functional groups demonstrating the high affinity for these click partners.

### Example 6—Size Exclusion Chromatography with Inductively Coupled Plasma Mass Spectrometry

Size exclusion chromatography (SEC) of intact antibodies coupled with inductively couple plasma mass spectrometry (SEC-ICP-MS) was used to confirm the reactivity of reactivity of the TCO functionalised 6E10 and 1E8 antibodies. SOD1 is a copper containing protein (32 kDa) used as a protein standard with a retention time of 315 seconds. Therefore, elution of a protein at 154 seconds is tentatively assigned as a full IgG (150 kDa). 1E8-PEG4-TCO (26 μg, 0.9 μM) was reacted with [CuL³] (24 of 10 mg/mL stock in DMSO, 10-fold excess of added TCO), centrifuged to remove any excess copper precipitate, then analysed via SEC-ICP-MS. From the chromatogram (see FIG. 6) it is possible to see that the 1E8-PEG4-TCO incubated with [CuL³] (pink) has significantly higher number of counts per second (CPS) at an antibody retention time, demonstrating that copper was binding to the antibody. To ensure that the copper complex was not binding to hydrophobic pockets within the antibody, unmodified 1E8 (10 μg, 1.3 μM) was incubated with [CuL³] (0.5 μL, 10 mg/mL) under the same conditions. This shows only baseline copper levels (blue) at 154 seconds in the antibody sample. The excess unbound, small molecule [CuL³] elutes at approximately 400 seconds.

### Example 7—IHC Brainstaining and Laser Ablation Inductively Coupled Mass Spectrometry

To ensure that unconjugated 6E10 and 1E8 antibodies selectivity bound to senile plaques, AD brain slides were incubated with 6E10 (1:1500 dilution) and 1E8 (1:500 dilution) and visualised with immunohistochemical staining techniques. Each antibody has a slightly different antigen binding site, both have a higher binding affinity for senile plaques over monomeric or fibrillar Aβ. 1E8 binds to senile plaques (antigen) and is specific for the first two amino acids of the Aβ peptide amino terminus, whereas 6E10 is reactive to amino acids 1-16 of Aβ and the epitope lies within amino acids 3-8 (EFRHDS). To confirm that the addition of hydrophilic PEG4-TCO functional groups did not interfere with the antigen binding sites on amyloid plaques, or cause aggregation of the protein due to the hydrophilic PEG linker, 6E10-PEG4-TCO was incubated and visualised (1:750, see FIG. 6). Staining showed that conjugation of the antibody with TCO functional groups did not alter binding affinity; serially stained sections of tissue correlated well to plaque distribution and chemically modified 6E10-PEG4-TCO stained both dense and more diffuse plaque formations.

1.-25. (canceled)

26. A method for the in vivo or ex vivo diagnostic imaging of a neurodegenerative disease or disorder, the method comprising sequentially administering to a patient or cell sample:
i) an antibody associated with the neurodegenerative disease or disorder, which has been modified to comprise a click-receptive dienophile; and
ii) a compound of Formula (I) or a pharmaceutically acceptable salt thereof; 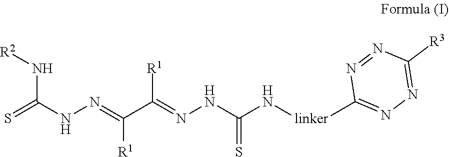
wherein:
each R¹ is independently selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R² is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R³ is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl; and
the linker is selected from the group consisting of optionally substituted alkylene, optionally substituted alkenylene, optionally substituted alkynylene, optionally substituted arylene, optionally substituted benzylene and heteroarylene; and
wherein the compound of Formula (I) is further complexed with a radioisotope and wherein the modified antibody covalently reacts in a click type reaction with the tetrazine of the compound of Formula (I) in situ.

27. The method according to claim 26, wherein the neurodegenerative disease or disorder is characterised by the presence of a protein aggregate associated with the neurodegenerative disease or disorder.

28. The method according to claim 27 wherein the protein aggregate is selected from a neurofibrillary tangle, a hyperphosphorylated tau protein, a Lewy body, aggregated alpha-synuclein, or aggregated TDP-43 protein.

29. The method according to claim 26, wherein the neurodegenerative disease or disorder is Alzheimer's disease or a brain cancer.

30. A method for the treatment of a neurodegenerative disease or disorder, the method comprising sequentially administering to a patient an effective amount of:
i) an antibody associated with the neurodegenerative disease or disorder which has been modified to comprise a click-receptive dienophile; and
ii) a compound of Formula (I) or a pharmaceutically acceptable salt thereof; 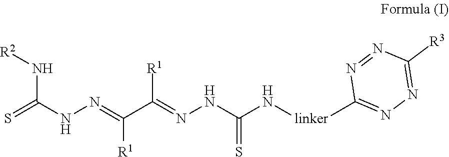
wherein:
each R¹ is independently selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R² is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R³ is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl; and
the linker is selected from the group consisting of optionally substituted alkylene, optionally substituted alkenylene, optionally substituted alkynylene, optionally substituted aryl ene, optionally substituted benzylene and heteroarylene;
wherein the compound of Formula (I) is further complexed with a radioisotope and wherein the modified antibody covalently reacts in a click-type reaction with the tetrazine of the compound of Formula (I) in situ.

31. The method according to claim 30, wherein the neurodegenerative disease or disorder is characterised by the presence of a protein aggregate associated with the neurodegenerative disease or disorder.

32. The method according to claim 31 wherein the protein aggregate is selected from a neurofibrillary tangle, a hyperphosphorylated tau protein, a Lewy body, aggregated alpha-synuclein, or aggregated TDP-43 protein.

33. The method according to claim 30, wherein the neurodegenerative disease or disorder is Alzheimer's disease or a brain cancer.

34. A compound of Formula (I), or a pharmaceutically acceptable salt thereof: 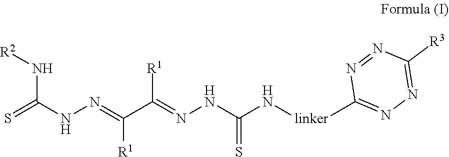
wherein:
each R¹ is independently selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R² is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl;
R³ is selected from the group consisting of H, optionally substituted C₁-C₁₂ alkyl, optionally substituted C₂-C₁₂ alkenyl, optionally substituted C₂-C₁₂ alkynyl, optionally substituted C₂-C₁₂ heteroalkyl, optionally substituted C₃-C₁₂ cycloalkyl, optionally substituted C₂-C₁₂ heterocycloalkyl, optionally substituted C₆-C₁₈ aryl and optionally substituted C₅-C₁₈ heteroaryl; and
the linker is selected from the group consisting of optionally substituted alkylene, optionally substituted alkenylene, optionally substituted alkynylene, optionally substituted arylene, optionally substituted benzylene and optionally substituted heteroarylene.

35. The compound according to claim 34, wherein each R1 is independently an optionally substituted alkyl group.

36. The compound according to claim 34, wherein R2 is an optionally substituted alkyl group.

37. The compound according to claim 34, wherein the linker is an optionally substituted benzylene group.

38. The compound according to claim 34, wherein R3 is H, or Me.

39. A metal complex having the structure of Formula (Ia): 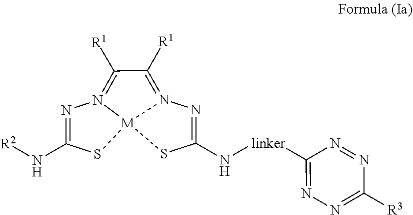
wherein M is a metal ion and R¹, R², R³ and the linker is selected from the group consisting of optionally substituted alkylene, optionally substituted alkenylene, optionally substituted alkynylene, optionally substituted arylene, optionally substituted benzylene and optionally substituted heteroarylene.

40. The metal complex according to claim 39, wherein M is selected from the group consisting of Cu, Tc, Gd, Ga, In, Co, Re, Fe, Au, Ag, Rh, Pt, Bi, Cr, W, Ni, V, Ir, Zn, Cd, Mn, Ru, Pd, Hg and Ti.

41. The metal complex of claim 39, wherein M is a copper ion.

42. The metal complex of claim 41, wherein the copper ion is a radioisotope of copper.

43. The metal complex of claim 42, wherein the radioisotope is selected from ⁶⁴Cu and ⁶⁷Cu.
